(19) United States
(12) Patent Application Publication
(10) Pub. No.: US 2007/0183997 A9
(43) Pub. Date: Aug. 9, 2007

(54) Composition comprising particles of at least one polymer dispersed in at least one fatty phase and at least one apolar oil

(76) Inventors: Caroline Lebre, Thiais (FR); Bertrand Lion, Paris (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/147,352

(22) Filed: Jun. 8, 2005

Related U.S. Application Data
(60) Provisional application No. 60/580,363, filed on Jun. 18, 2004.

(30) Foreign Application Priority Data
Jun. 8, 2004 (FR) 04 06172

(51) Int. Cl. A61K 7/021; A61K 7/025

(52) U.S. Cl. 424/63; 424/64

(57) ABSTRACT

The present disclosure relates to a cosmetic composition comprising particles of at least one polymer dispersed in a fatty phase, the at least one fatty phase is free of volatile oil or comprises less than 50% by weight of volatile oil relative to the weight of the at least one fatty phase, and the liquid fatty phase comprising at least 5% by weight of at least one sparingly polar or apolar oil. The apolar or sparingly polar oil may be, for example, a non-volatile hydrocarbon-based apolar oil.

This application claims benefit of U.S. Provisional Application No. 60/580,363, filed Jun. 18, 2004, the contents of which are incorporated herein by reference. This application also claims benefit of priority under 35 U.S.C. § 119 to French Patent Application No. 04 06172, filed Jun. 8, 2004, the contents of which are also incorporated by reference.

In one aspect of the present disclosure, the disclosure relates to a care or makeup composition for keratin materials, such as the skin of either the human face or body, including the scalp, the integuments, for instance the eyelashes, the eyebrows, the nails and the hair, and also the lips and the eyelids, which composition comprises particles of at least one polymer dispersed in at least one fatty phase.

European Patent Application No. EP 1 002 528 discloses transfer-resistant compositions comprising a dispersion of polymer particles in a volatile oil. These compositions predominantly contain a volatile oil, and also non-volatile silicone oils and non-volatile hydrocarbon-based oils. The non-volatile hydrocarbon-based oils are in very small amount, for example, 3%.

There is still a need for a cosmetic product that is glossy and that has good staying power. The inventors have found, surprisingly, that compositions comprising particles of at least one polymer dispersed in at least one fatty phase wherein the at least one fatty phase comprises no more than 50% by weight of a volatile oil and wherein the at least one fatty phase comprises at least 5% of at least one apolar or sparingly polar oil, according to at least one embodiment of the present disclosure can result in good level of gloss while at the same time good staying power may also be obtained.

In addition, the compositions of the prior art with good gloss often have the drawback of little staying power. However, women are nowadays in search of products, for example, lip or eyelid makeup products, which have a good level of gloss and also good staying power. The glossy composition according to at least one embodiment of the present disclosure may have good staying power.

One embodiment of the present disclosure may be, for example, a hair product or a makeup product for the human body, lips or integuments that has care and/or treatment properties. Further, for example, an embodiment of the disclosure may be chosen from lipsticks or lip glosses, eyeshadows, tattoo products, mascaras, eyeliners, artificial skin tanning products, optionally tinted care creams or protective creams, hair dye products and haircare products

According to one embodiment, the composition according to the disclosure may have at least one of the following attributes: good staying power, non-tackiness, and non-migrating outside the initial deposit. For example, in one embodiment, when the composition according to the disclosure is applied to the skin or the lips, it may afford a sensation of comfort on application and once deposited.

One embodiment of the present disclosure is, a cosmetic composition comprising particles of at least one polymer dispersed in at least one fatty phase,
wherein the at least one fatty phase comprises no more than 50% by weight of volatile oil, relative to the weight of the at least one fatty phase, and
wherein the at least one fatty phase comprises at least 5% by weight of at least one sparingly polar or apolar oil, relative to the weight of the at least one fatty phase.

In another embodiment, the at least one fatty phase is free of volatile oil.

In another embodiment, the at least one fatty phase may, for example, comprise at least 10% by weight of at least one apolar or sparingly polar oil.

In another embodiment, the mean gloss at 20° of a deposit of a composition according to the disclosure spread onto a support is no less than 30 out of 100.

As used herein, the term “mean gloss” means the gloss as may be conventionally measured using a glossmeter by the following method:
(A) A coat ranging from 50 μm to 150 μm in thickness of the composition is spread using an automatic spreader onto a Leneta brand contrast card of reference Form 1A Penopac. The coating covers at least the white background of the card. When the composition is solid, it is melted, if necessary, on the card after it has been spread and has covered the white background.
(B) The deposit is left to dry for 24 hours at a temperature of 30° C., and the gloss at 200 is then measured on the white background using a Byk Gardner brand glossmeter of reference microTri-Gloss.
(C) This measurement (between 0 and 100) is repeated at least three times, and the mean gloss is the mean of the at least three measurements taken.

The mean gloss of the composition spread onto a support measured at 200 may be, for example, greater than or equal to 30 out of 100, greater than or equal to 35 out of 100, greater than or equal to 40 out of 100, greater than or equal to 45 out of 100, greater than or equal to 50 out of 100, greater than or equal to 55 out of 100, or greater than or equal to 60 out of 100.

In another embodiment, the mean gloss of the composition spread onto a support measured at 600, may be, for example, greater than or equal to 50 out of 100, greater than or equal to 60 out of 100, greater than or equal to 65 out of 100, greater than or equal to 70 out of 100, greater than or equal to 75 out of 100, greater than or equal to 80 out of 100, greater than or equal to 85 out of 100 or greater than or equal to 90 out of 100.

The mean gloss at 60° is measured as described above, by taking the measurement at 60° rather than at 20°.

According to another embodiment, the mean gloss at 20° of a deposit of the composition spread onto a support may be, for example, no less than 35 out of 100 and the mean gloss at 60° of a deposit of the composition spread onto a support may be, for example, no less than 65 out of 100. In this embodiment, the composition may, for example, be used in a liquid lipstick.

The present disclosure also relates to a cosmetic composition wherein the composition improves the gloss of a deposit on the skin and/or the lips and/or the integuments, for example, a makeup.

As used herein, the term “fatty phase” means any non-aqueous medium.

In an embodiment, the at least one fatty phase may be, for example, at least partially liquid at room temperature (25° C.) and atmospheric pressure (760 mmHg) and is composed of at least one fatty substance that is liquid at room temperature, for example, oils.

The present disclosure also relates to a cosmetic composition wherein the composition gives gloss and migration resistance to a deposit on the skin and/or the lips and/or the integuments, for example, a makeup deposit.

At Lease One Polymer in Dispersion

According to the present disclosure, the at least one polymer may be a solid that is insoluble in the at least one fatty phase of the composition at room temperature, for example at 25° C. The at least one polymer may also, for example, be insoluble in the at least one fatty phase at its softening point. For example, the at least one polymer is not a wax which may be soluble in the at least one fatty phase at a temperature above its melting point.

The particles of at least one polymer in a dispersion may allow, for example, the formation of a deposit that is continuous and homogeneous and/or is characterized by the overlapping of the polymer chains.

In an embodiment, the composition according to the present disclosure may, for example, comprise at least one stable dispersion of essentially spherical polymer particles of at least one polymer, in the at least one fatty phase.

In another embodiment, the dispersions may be, for example, in the form of polymer nanoparticles in stable dispersion in a liquid organic phase. The nanoparticles may have, for example, a mean size ranging from 5 to 800 nm, for example, ranging from 50 to 500 nm. In another embodiment, the polymer particles may range up to 1 μm in size.

For example, the particles of at least one polymer in dispersion are insoluble in water-soluble alcohols, for example, ethanol.

In another embodiment, the particles of at least one polymer in dispersion may have, for example, a molecular weight ranging from 2000 to 10,000,000 g/mol, and a Tg ranging from −100° C. to 300° C., for example, from −50° C. to 100° C. and further, for example, from −10° C. to 50° C.

In another embodiment, the polymer may be film-forming, for example, film-forming polymers with a low Tg, of less than or equal to skin temperature, for example, less than or equal to 40° C.

As used herein, the term “film-forming polymer” means a polymer that is capable, by itself or in the presence of an auxiliary film-forming agent, of forming a continuous film that adheres to a support, for example, to keratin materials, for example, forming a cohesive film, and further, for example, forming a film which may optionally be isolated from the support.

In another embodiment, the polymer may be a non-film-forming polymer. As used herein, the term “non-film-forming polymer” means a polymer that is incapable by itself of forming an isolable film.

Among the film-forming polymers that may be used herein, non-limiting mention may be made of acrylic or vinyl free-radical homopolymers or copolymers, for example, with a Tg of less than or equal to 40° C. and further, for example, ranging from −10° C. to 30° C., used alone or as a mixture.

Among the non-film-forming polymers that may be used herein, non-limiting mention may be made of optionally crosslinked vinyl or acrylic free-radical homopolymers or copolymers, for example, with a Tg of greater than 40° C. and further, for example, 45° C. to 150° C., used alone or as a mixture.

As used herein, the term “free-radical polymer” means a polymer obtained by polymerization of unsaturated monomers, for example, ethylenic monomers, each monomer is capable of homopolymerizing (unlike polycondensates). The free-radical polymers may, for example, be vinyl polymers or copolymers, for example, acrylic polymers.

The acrylic polymers may result from the polymerization of ethylenically unsaturated monomers comprising at least one acid group and/or esters of these acid monomers and/or amides of these acids.

Meth(acrylic)/(meth)acrylate copolymers, for example, acrylic/acrylate copolymers wherein the mass ratio of the acrylic units and of the acrylate units ranges from 0.1% to 40%, for example, from 2% to 30% and further, for example, from 5% and 20%.

Monomers bearing an acid group may be chosen from α,β-ethylenic unsaturated carboxylic acids such as acrylic acid, (meth)acrylic acid, crotonic acid, maleic acid or itaconic acid. (Meth)acrylic acid and crotonic acid, for example, (meth)acrylic acid.

The acid monomer esters may be, for example, chosen from (meth)acrylic acid esters (also known as (meth)acrylates), for instance alkyl (meth)acrylates, for example, of a C₁-C₂₀, for example, C₁-C₈ alkyl, aryl (meth)acrylates, for example, of a C₆-C₁₀ aryl, and hydroxyalkyl (meth)acrylates, for example, of a C₂-C₆ hydroxyalkyl. Alkyl (meth)acrylates may be chosen from methyl, ethyl, butyl, isobutyl, 2-ethylhexyl and lauryl (meth)acrylate. Hydroxyalkyl (meth)acrylates may be chosen from hydroxyethyl (meth)acrylate and 2-hydroxypropyl (meth)acrylate. Aryl (meth)acrylates that may be, for example, mentioned are benzyl or phenyl acrylate.

The (meth)acrylic acid esters may be, for example, chosen from the alkyl (meth)acrylates.

Free-radical polymers may be, for example, chosen from copolymers of (meth)acrylic acid and of alkyl (meth)acrylate, for example, of a C₁-C₄ alkyl. Methyl acrylates optionally copolymerized with acrylic acid may, for example, be used.

Amides of the acid monomers may be, for example, chosen from (meth)acrylamides, for example, N-alkyl(meth)acrylamides, for example, of a C₂-C₁₂ alkyl, such as N-ethylacrylamide, N-t-butylacrylamide and N-octylacrylamide, for example, N-di(C₁-C₄)alkyl(meth)acrylamides.

The acrylic polymers may also result from the polymerization of ethylenically unsaturated monomers comprising at least one amine group, in free form or in partially or totally neutralized form, or in partially or totally quaternized form. Such monomers may be, for example, dimethylaminoethyl (meth)acrylate, dimethylaminoethyl(meth)acrylamide, vinylamine, vinylpyridine or diallyidimethylammonium chloride.

The vinyl polymers may also result from the homopolymerization or copolymerization of at least one monomer chosen from vinyl esters and styrene monomers. For example, these monomers may be polymerized with acid monomers and/or esters thereof and/or amides thereof, such as those mentioned previously. Vinyl esters may be, for example, chosen from vinyl acetate, vinyl propionate, vinyl neodecanoate, vinyl pivalate, vinyl benzoate and vinyl t-butylbenzoate. Styrene monomers may be, for example, chosen from styrene and α-methylstyrene.

The list of monomers given is not limiting, and it is possible to use any monomer known to those skilled in the art which may be in the categories of acrylic and vinyl monomers (for example, monomers modified with a silicone chain).

As other vinyl monomers that may be used, non-limiting mention may also be made of:
N-vinylpyrrolidone, N-vinylcaprolactam, vinyl-N-(C₁-C₈)alkylpyrroles, vinyloxazoles, vinylthiazoles, vinylpyrimidines and vinylimidazoles,
olefins such as ethylene, propylene, butylene, isoprene or butadiene.

The vinyl polymer may be crosslinked with at least one difunctional monomers for example, comprising at least two ethylenic unsaturations, such as ethylene glycol di(meth)acrylate or diallyl phthalate.

In a non-limiting manner, the at least one polymer in dispersion of the present disclosure may be chosen from the following polymers or copolymers: polyurethanes, poly-urethane-acrylics, polyureas, polyurea-polyurethanes, polyester-polyurethanes, polyether-polyurethanes, polyesters, polyesteramides, alkyds; acrylic and/or vinyl polymers or copolymers; acrylic-silicone copolymers; polyacrylamides; silicone polymers, for instance silicone polyurethanes or silicone acrylics; fluoro polymers, and mixtures thereof.

The at least one polymer in dispersion in the at least one fatty-phase may be present in an amount ranging from 5% to 40%, for example, from 5% to 35% and further, for example, from 8% to 30% of the weight of solids in the composition.

According to one embodiment, the at least one polymer particles in dispersion are surface-stabilized with at least one stabilizer that is solid at room temperature. In this case, the amount of solids in the dispersion is the total amount of polymer+stabilizer, given that the amount of polymer cannot be less than 5%.

For example, a dispersion of particles of at least one film-forming polymer may be used.

At Least One Stabilizer

The polymer particles in organic medium may be, for example, surface-stabilized, as the polymerization proceeds, by means of at least one stabilizer that may be a block polymer, a grafted polymer and/or a random polymer, alone or as a mixture. The stabilization may take place by any known means, and for example, by direct addition of the block polymer, grafted polymer and/or random polymer during the polymerization.

The at least one stabilizer is, for example, also present in the mixture before polymerization. However, it is also possible to add it continuously, for example, when the monomers are also added continuously.

The at least one stabilizer may be present in an amount ranging from 2 to 30% by weight, for example, 5 to 20% by weight of stabilizer, relative to the initial monomer mixture.

When a grafted polymer and/or a block polymer is used as a stabilizer, the synthesis solvent is chosen wherein at least some of the grafts or blocks of the polymer-stabilizer are soluble in the solvent, the rest of the grafts or blocks are insoluble therein. The polymer-stabilizer used during the polymerization should be soluble, or dispersible, in the synthesis solvent. Furthermore, a stabilizer whose insoluble blocks or grafts have a certain affinity for the polymer formed during the polymerization may be used.

Among the grafted polymers that may be mentioned are silicone polymers grafted with a hydrocarbon-based chain; hydrocarbon-based polymers grafted with a silicone chain.

Grafted copolymers comprising, for example, an insoluble skeleton of polyacrylic type with soluble grafts of poly(12-hydroxystearic acid) type are also suitable for use.

Thus, grafted-block or block copolymers comprising at least one block of polyorganosiloxane type and at least one block of a free-radical polymer, for instance grafted copolymers of acrylic/silicone type, may thus be used, for example, when the synthesis medium and then the organic phase of the first composition comprises a silicone phase.

It is also possible to use grafted-block or block copolymers comprising at least one block of polyorganosiloxane type and at least one block of a polyether. The polyorganosiloxane block may, for example, be a polydimethylsiloxane or a poly(C₂-C₁₈)alkylmethylsiloxane; the polyether block may be a poly(C₂-C₁₈)alkylene, for example, polyoxyethylene and/or polyoxypropylene. For example, dimethicone copolyols or (C₂-C₁₈)alkyldimethicone copolyols such as those sold under the name “Dow Corning 3225C” by the company Dow Corning, and lauryl methicones such as those sold under the name “Dow Corning Q2-5200” by the company Dow Corning, may be used.

Grafted-block or block copolymers may be, for example, chosen from those comprising at least one block resulting from the polymerization of at least one ethylenic monomer comprising at least one optionally conjugated ethylenic bonds, for instance ethylene or dienes such as butadiene and isoprene, and of at least one block of a vinyl polymer, for example, a styrene polymer. When the ethylenic monomer comprises several optionally conjugated ethylenic bonds, the residual ethylenic unsaturations after the polymerization are, for example, hydrogenated. Thus, in a known manner, the polymerization of isoprene leads, after hydrogenation, to the formation of an ethylene-propylene block, and the polymerization of butadiene leads, after hydrogenation, to the formation of an ethylene-butylene block. Among these polymers that may be mentioned are block copolymers, for example, of “diblock” or “triblock” type such as polystyrene/polyisoprene (SI), polystyrene/polybutadiene (SB) such as those sold under the name “Luvitol HSB” by BASF, of the type such as polystyrene/copoly(ethylene-propylene) (SEP) such as those sold under the name “Kraton” by Shell Chemical Co. or of the type such as polystyrene/copoly(ethylene-butylene) (SEB). Kraton G1650 (SEBS), Kraton G1651 (SEBS), Kraton G1652 (SEBS), Kraton G1657X (SEBS), Kraton G1701X (SEP), Kraton G1702X (SEP), Kraton G1726X (SEB), Kraton D-1101 (SBS), Kraton D-1102 (SBS) and Kraton D-1107 (SIS) may be used, for example. The polymers are, for example, known as hydrogenated or non-hydrogenated diene copolymers.

Gelled Permethyl 99A-750, 99A-753-59 and 99A-753-58 (mixture of triblock and of star polymer), Versagel 5960 from Penreco (triblock+star polymer); OS129880, OS129881 and OS84383 from Lubrizol (styrene/(meth)acrylate copolymer) may also be used.

As grafted-block or block copolymers comprising at least one block resulting from the polymerization of at least one ethylenic monomer comprising at least one ethylenic bond and of at least one block of an acrylic polymer, non-limiting mention may be made of poly(methyl (meth)acrylate)/polyisobutylene diblock or triblock copolymers or grafted copolymers comprising a poly(methyl (meth)acrylate) skeleton and polyisobutylene grafts.

As grafted-block or block copolymers comprising at least one block resulting from the polymerization of at least one ethylenic monomer comprising at least one ethylenic bond and of at least one block of a polyether such as a C₂-C₁₈ polyalkylene (for example, polyethylene and/or polyoxypropylene), non-limiting mention may be made of polyoxyethylene/polybutadiene or polyoxyethylene/polyisobutylene diblock or triblock copolymers.

When a random polymer is used as the at least one stabilizer, it is chosen wherein it has a sufficient amount of groups making it soluble in the intended organic synthesis medium.

Copolymers based alkyl acrylates or (meth)acrylates derived from C₁-C₄ alcohols and on alkyl acrylates or (meth)acrylates derived from C₈-C₃₀ alcohols may thus be used. Non-limiting mention may be made for example, of stearyl (meth)acrylate/methyl (meth)acrylate copolymer.

When the synthesis medium is apolar, the at least one stabilizer, for example, may be a polymer that provides the fullest possible coverage of the particles, several polymer-stabilizer chains then are absorbed onto a particle of polymer obtained by polymerization.

For example, the at least one stabilizer may be either a grafted polymer or a block polymer, so as to have better interfacial activity. For example, blocks or grafts that are insoluble in the synthesis solvent provide bulkier coverage at the surface of the particles.

When the liquid synthesis solvent comprises at least one silicone oil, the at least one stabilizer may be, for example, chosen from grafted-block and block copolymers comprising at least one block of polyorganosiloxane type and at least one block of a free-radical polymer and of a polyether or of a polyester, for instance polyoxypropylene and/or polyoxyethylene blocks.

When the liquid organic phase does not comprise any silicone oil, the at least one stabilizer may, for example, be chosen from:
(a) grafted-block or block copolymers comprising at least one block of polyorganosiloxane type and at least one block of a free-radical polymer or of a polyether or a polyester,
(b) copolymers of alkyl acrylates or (meth)acrylates derived from C₁-C₄ alcohols and of alkyl acrylates or (meth)acrylates derived from C₈-C₃₀ alcohols,
(c) grafted-block or block copolymers comprising at least one block resulting from the polymerization of at least one ethylenic monomer comprising conjugated ethylenic bonds, and at least one block of a vinyl or acrylic polymer or of a polyether or of a polyester, or mixtures thereof.

Diblock polymers may be, for example, used as the at least one stabilizer.

At Least One Plasticizer

When the polymer has a glass transition temperature that is too high for the intended application, at least one plasticizer may be combined therewith. The at least one plasticizer may be chosen from the plasticizers usually used in the field of application and, for example, from compounds liable to be solvents for the polymer. Coalescers may also be used in order to aid the polymer to form a continuous and homogeneous deposit.

The coalescers or plasticizers that may be used in the present disclosure are, for example, those mentioned in document FR-A-2 782 917.

The composition according to the present disclosure may comprise at least one ester of at least one carboxylic acid comprising from 1 to 7 carbon atoms and of a polyol comprising at least four hydroxyl groups, the ester comprising a molar mass of less than 5000 g/mol.

The polyol may be a monosaccharide or a polysaccharide comprising from 1 to 10 saccharides, for example, from 1 to 4 and further, for example, from one or two saccharides.

The polyol according to the present disclosure may be, for example, a disaccharide. Among the disaccharides that may be mentioned are sucrose (α-D-glucopyranosyl-(1-2)-β-D-fructofuranose), lactose (β-D-galactopyranosyl-(1-4)-β-D-glucopyranose) and maltose (α-D-glucopyranosyl-(1-4)-β-D-glucopyranose).

According to one embodiment, the ester is sucrose diacetate hexakis(2-methylpropanoate).

The ester may be, for example, liquid at room temperature and atmospheric pressure. It may be, for example, present in an amount ranging from 0.1% to 25% by weight, for example, from 0.5% to 15% by weight and further, for example, from 3% to 15% by weight.

The mass ratio between the polymer particles and the ester of acid and polyol may, for example, range from 0.5 to 100, for example, from 1 to 50, further, for example, from 1 to 10 and even further, for example, from 1 to 5.

At Least One Fatty Phase

The at least one fatty phase of the composition may, for example, comprise at least one cosmetically or dermatologically acceptable and physiologically acceptable oil, chosen, for example, from carbon-based oils, hydrocarbon-based oils, fluoro oils and/or silicone oils of mineral, plant or synthetic origin, alone or as a mixture.

As used herein, the term “oil” means any non-aqueous medium that is liquid at room temperature (25° C.) and atmospheric pressure (760 mmHg).

The total fatty phase of the composition may be present in an amount ranging from 5% to 90%, for example, from 20% to 85% of the total weight of the composition. The total fatty phase of the composition may be present in an amount of at least 30% of the total weight of the composition.

According to one embodiment, the at least one fatty phase is free of or comprises less than 50% by weight of at least one volatile oil, relative to the total weight of the at least one fatty phase. For example, the fatty phase comprises less than 40%, for example, less than 30%, further, for example, less than 20% and even further, for example, less than 10% by weight of at least one volatile oil, relative to the total weight of the at least one fatty phase.

At Least One Apolar or Sparingly Polar Oil

According to one embodiment, the at least one fatty phase comprises at least one apolar or sparingly polar oil, which is present in an amount of at least 5% by weight, relative to the total weight of the composition. For example, the oil of the at least one fatty phase may be non-volatile hydrocarbon-based apolar or sparingly polar oils, which may be hydrocarbon-based.

For example, the apolar oils have a solubility parameter a =°.

As used herein, the term “polar oil” means an oil composed of chemical compounds comprising at least one polar group. The “polar groups” are well known to those skilled in the art; they may be, for example, ionic polar groups or nonionic groups chosen from —COOH; —OH; ethylene oxide; propylene oxide; —PO₄; —NHR; —NR₁R₂ with R₁ and R₂ optionally forming a ring and representing a linear or branched C₁ to C₂₀ alkyl or alkoxy radical.

The sparingly polar oils comprise oils that have a mean solubility parameter at 25° C. of: 0<δₐ<5.0 (J/cm³)^{1/2}.

The highly polar oils have a mean solubility parameter δₐ according to the Hansen solubility space, at 25° C., of: δₐ≧5.0 (J/cm³)^{1/2}.

The definition and calculation of the solubility parameters in the three-dimensional Hansen solubility space are described in the article by C. M. Hansen: “The three dimensional solubility parameters” J. Paint Technol. 39, 105 (1967).

According to this Hansen space:
δ_{D} is the London dispersion forces derived from the formation of dipoles induced during molecular impacts;
δₚ is the Debye interaction forces between permanent dipoles and the Keesom interaction forces between induced dipoles and permanent dipoles;
δₕ is the specific interaction forces (such as hydrogen bonding, acid/base, donor/acceptor, etc.);
δₐ is determined by the equation: δₐ=(δₚ²+δₕ²)^{1/2}.

The parameters δₚ, δₕ, δ_{D} and δₐ are expressed in (J/cm³)^{1/2}.

When the oily phase is a mixture of different oils, the solubility parameters of the mixture are determined from those of the compounds taken separately, according to the following relationships:
δ_{Dmixt}=Σ xi δ_{Di}; δₚₘᵢₓₜ=Σ xi δₚᵢ and δₕₘᵢₓₜ=Σ xi δₕᵢ
δₐₘᵢₓₜ=(δ²ₚₘᵢₓₜ+δ²ₕₘᵢₓₜ)^{1/2}
wherein xi represents the volume fraction of compound i in the mixture.

It is within the capability of a person skilled in the art to determine the amounts of each oil to obtain an oily phase that satisfies the desired criteria.

The at least one apolar or sparingly polar oil may be, for example, hydrocarbon-based. As used herein, the term “hydrocarbon-based oil” means an oil formed essentially from, or comprising, carbon and hydrogen atoms, and optionally oxygen and nitrogen atoms, and comprising no silicon or fluorine atoms. It may comprise alcohol, ester, ether, carboxylic acid, amine and/or amide groups.

The at least one apolar or sparingly polar oil may be, for example, non-volatile. As used herein, the term “non-volatile oil” means any oil with a non-zero vapor pressure at room temperature and atmospheric pressure, of less than 0.02 mmHg, for example, less than 10⁻³ mmHg.

The at least one apolar or sparingly polar oil may be, for example, present in an amount of at least 5% by weight, relative to the total weight of the composition. The at least one apolar or sparingly polar oil may be present in an amount ranging from 5% to 80% by weight, relative to the total weight of the composition, for example, from 10% to 60% by weight and further, for example, from 10% to 40% by weight, relative to the total weight of the composition.

When hydrocarbon-based, the at least one apolar or sparingly polar oil may be present in an amount ranging from 10% to 40% by weight, for example, from 15% to 30% by weight, relative to the total weight of the composition.

For example, the a least one apolar or sparingly polar oil may be a non-volatile apolar hydrocarbon-based oil.

According to one embodiment, the apolar hydrocarbon-based oil is free of hetero atom(s). As used herein, the term “hetero atom” means an atom other than carbon or hydrogen.

According to one embodiment, the non-volatile hydrocarbon-based apolar oil may be, for example, chosen from linear or branched saturated alkanes.

The non-volatile apolar or sparingly polar hydrocarbon-based oil may be chosen from hydrocarbon-based oils with a molar mass ranging from 300 to 900 g/mol, for example, from 350 to 800 g/mol.

According to one embodiment, the non-volatile hydrocarbon-based apolar oil is chosen from linear or branched hydrocarbons such as liquid paraffin, liquid petroleum jelly and liquid naphthalene, hydrogenated polyisobutene, isoeicosane, squalane and decene/butene copolymers, and mixtures thereof.

According to one embodiment, the at least one fatty phase comprises from 30% to 70% by weight of apolar non-volatile hydrocarbon-based oil(s), relative to the weight of the at least one fatty phase, for example from 40% to 60% by weight, relative to the weight of the at least one fatty phase.

As other examples of non-volatile apolar or sparingly polar oils, non-limiting mention may be made of:
hydrocarbon-based oils of animal origin, for instance squalene;
hydrocarbon-based plant oils such as liquid triglycerides of fatty acids of at least 10 carbon atoms;
synthetic esters and ethers, for example, of fatty acids, for instance the oils of formula R₁(CO)ₓOR₂ wherein R₁ is chosen from an acid residue comprising from 2 to 29 carbon atoms wherein x is chosen from 0 or 1 and R₂ is chosen from a hydrocarbon-based chain comprising from 3 to 30 carbon atoms, for instance tributyl acetyl citrate, oleyl erucate, 2-octyldodecyl behenate, triisoarachidyl citrate, isocetyl or octyldodecanyl stearoylstearate, n-propyl acetate, tridecyl trimellitate, diisocetyl dodecanedioleate or stearate, arachidyl propionate, dibutyl phthalate, propylene carbonate or octyldodecyl pentanoate; polyol esters, for instance vitamin F, sorbitan isostearate, glyceryl triisostearate or diglyceryl triisostearate; and
mixtures thereof.

The at least one fatty phase of the composition may also comprise at least one non-volatile silicone oil chosen from:
polydimethylsiloxanes (PDMS) optionally comprising a C₃-C₄₀ alkyl or C₃-C₄₀ alkoxy chain or a phenyl radical; the polydimethylsiloxanes comprising phenyl radicals may be chosen from phenyl trimethicones;
optionally fluorinated polyalkylmethylsiloxanes, for instance polymethyltrifluoropropyldimethylsiloxanes,
polyalkylmethylsiloxanes substituted with functional groups such as hydroxyl, thiol and/or amine groups; and
polysiloxanes modified with fatty acids, fatty alcohols or polyoxyalkylenes.

According to one embodiment, the at least one fatty phase of the composition comprises less than 10% of at least one silicone oils. For example, the at least one fatty phase comprises less than 5%, less than 3%, or even less than 1% of at least one silicone oils.

At Least One Non-Volatile Highly Polar Oil

The at least one fatty phase may comprise, besides the apolar or sparingly polar oil as described above, at least one highly polar non-volatile oil chosen from fatty acid esters comprising from 7 to 29 carbon atoms, for instance diisostearyl malate, isopropyl palmitate, diisopropyl adipate, caprylic/capric acid triglycerides, for instance those sold by the company Stearineries Dubois or those sold under the names Miglyol 810, 812 and 818 by the company Dynamit Nobel, Shea butter oil, isopropyl myristate, butyl stearate, hexyl laurate, diisopropyl adipate, isononyl isononate, 2-hexyldecyl laurate, 2-octyldecyl palmitate, 2-octyldodecyl myristate or lactate, 2-diethylhexyl succinate, 2-ethylhexyl palmitate, 2-octyldodecyl stearate or castor oil; lanolic acid, lauric acid or stearic acid esters; higher fatty alcohols (comprising from 7 to 29 carbon atoms) such as stearyl alcohol, linoleyl alcohol, linolenyl alcohol, isostearyl alcohol, 2-octyidodecanol, decanol, dodecanol, octadecanol or oleyl alcohol; higher fatty acids (comprising from 7 to 29 carbon atoms) such as myristic acid, palmitic acid, stearic acid, behenic acid, oleic acid, linoleic acid, linolenic acid or isostearic acid; mixtures thereof.

These one or more non-volatile highly polar oils may be present in an amount ranging from 0.1% to 10%, for example, from 1% to 5% of the total weight of the composition.

At Least One Volatile Oil of the at Least One Fatty Phase

The at least one fatty phase of the composition according to the present disclosure may comprise at least one volatile oil, provided that the at least one volatile oil is present in an amount of less than 50% by weight of the at least one fatty phase. These oils may be hydrocarbon-based oils or silicone oils optionally comprising alkyl or alkoxy groups that are pendent or at the end of a silicone chain.

As used herein, the term “volatile oil” means any oil with a vapor pressure, at room temperature and atmospheric pressure, of greater than 0.02 mmHg.

As volatile silicone oils that may be used in the present disclosure, non-limiting mention may be made of linear or cyclic silicones with a viscosity at room temperature of less than 8 cSt and, for example, comprising from 2 to 7 silicon atoms, these silicones optionally comprising alkyl or alkoxy groups of 1 to 10 carbon atoms. As volatile silicone oils that may be used in the present disclosure, non-limiting mention may be made, for example, of octamethylcyclotetrasiloxane, decamethylcyclopentasiloxane, dodecamethylcyclohexasiloxane, heptamethylhexyltrisiloxane, heptamethyloctyltrisiloxane, octamethyltrisiloxane and decamethyltetrasiloxane, and mixtures thereof.

As volatile oils that may be used in the present disclosure, C₈-C₁₆ isoalkane oils (also known as isoparaffins), for instance isododecane, isodecane or isohexadecane and, for example, the oils sold under the trade names Isopar and Permethyl, and isododecane (Permethyl 99A), may be used.

For example, the at least one volatile oil may be present in an amount less than 10% of the at least one fatty phase of the composition disclosed herein, for example, less than 5%, less than 3% or even less than 1% of the at least one fatty phase of the composition disclosed herein.

At Least One Oil of High Molar Mass

According to one embodiment, the at least one fatty phase may comprise at least one oil of high molar mass, for example, ranging from 650 to 10,000 g/mol.

The composition according to the present disclosure may, for example, comprises from 2% to 30%, for example, from 5% to 25% or from 5% to 15% of at least one oil with a molar mass ranging from 650 to 10 000 g/mol, for example, ranging from 750 to 7500 g/mol.

The oil with a molar mass ranging from 650 to 10 000 g/mol may be chosen from:
polybutylenes such as Indopol H-100 (molar mass or MM=965 g/mol), Indopol H-300 (MM=1340 g/mol) and Indopol H-1500 (MM=2160 g/mol), sold or manufactured by the company Amoco,
hydrogenated polyisobutylenes such as Panalane H-300 E sold or manufactured by the company Amoco (M=1340 g/mol), Viseal 20 000 sold or manufactured by the company Synteal (MM=6000 g/mol) and Rewopal PIB 1000 sold or manufactured by the company Witco (MM=1000 g/mol),
polydecenes and hydrogenated polydecenes such as: Puresyn 10 (MM=723 g/mol) and Puresyn 150 (MM=9200 g/mol), sold or manufactured by the company Mobil Chemicals,
vinylpyrrolidone copolymers such as: the vinylpyrrolidone/1-hexadecene copolymer, Antaron V-216 sold or manufactured by the company ISP (MM=7300 g/mol),
esters such as:
a) linear fatty acid esters with a total carbon number ranging from 35 to 70, for instance pentaerythrityl tetrapelargonate (MM=697.05 g/mol),
b) hydroxylated esters such as polyglyceryl-2 triisostearate (MM=965.58 g/mol),
c) aromatic esters such as tridecyl trimellitate (MM=757.19 g/mol),
d) esters of branched C₂₄-C₂₈ fatty alcohol or fatty acids, such as those described in European Patent Application No. EP-A-0 955 039, and for example, triisoarachidyl citrate (MM=1033.76 g/mol), pentaerythrityl tetraisononanoate (MM=697.05 g/mol), glyceryl triisostearate (MM=891.51 g/mol), glyceryl tris(2-decyl)tetradecanoate (MM=1143.98 g/mol), pentaerythrityl tetraisostearate (MM=1202.02 g/mol), polyglyceryl-2 tetraisostearate (MM=1232.04 g/mol) or pentaerythrityl tetrakis(2-decyl)tetradecanoate (MM=1538.66 g/mol),
e) diol dimer esters and polyesters, such as esters of a diol dimer and of a fatty acid, and esters of a diol dimer and of a diacid.
The esters of a diol dimer and of a monocarboxylic acid may be obtained from a monocarboxylic acid comprising from 4 to 34 carbon atoms, for example, from 10 to 32 carbon atoms, these acids are linear or branched, and saturated or unsaturated.
As illustrative examples of monocarboxylic acids that are suitable for the present disclosure, mention may be made of fatty acids.
The esters of a diol dimer and of a dicarboxylic acid may be obtained from a diacid dimer derived for example, from the dimerization of an unsaturated fatty acid, for example, of C₈ to C₃₄, of C₁₂ to C₂₂, of C₁₈ to C₂₀ and of C₁₈.
According to one embodiment, the esters may be the diacid dimer from which the diol dimer to be esterified is also derived.
The diol dimer esters may be obtained from a diol dimer produced by catalytic hydrogenation of a diacid dimer as described above, for example hydrogenated dilinoleic diacid.
As illustrations of diol dimer esters, non-limiting mention may be made of the esters of dilinoleic diacids and of dilinoleyl diol dimers sold by the company Nippon Fine Chemical under the trade name Lusplan DD-DA5® and DD-DA7®.
silicone oils such as phenylsilicones, for instance Belsil PDM 1000 from the company Wacker (MM=9000 g/mol),
oils of plant origin such as sesame oil (820.6 g/mol), and
mixtures thereof.
   At Least One Wax, Pasty Compound

The composition according to the present disclosure may comprise at least one wax.

As used herein, the term “wax” is a lipophilic fatty compound that is solid at room temperature (25° C.), which undergoes a reversible solid/liquid change of state, with a melting point of greater than or equal to 30° C., and with an anisotropic crystal organization in the solid state. The crystals diffract and/or scatter light, giving the composition a cloudy, more or less opaque appearance. By bringing the wax to its melting point, it is possible to make it miscible with oils and to form a microscopically homogeneous mixture, but, on returning the temperature of the mixture to room temperature, recrystallization of the wax in the oils of the mixture is obtained.

As used herein, the melting point of the wax corresponds to the temperature of the most endothermic peak observed by thermal analysis (DSC) as described in standard ISO 11357-3; 1999.

The melting point of the wax may be measured using a differential scanning calorimeter (DSC), for example the calorimeter sold under the name MDSC 2920 by the company TA Instruments.

The measuring protocol is, for example, as follows:

A sample of 5 mg of wax placed in a crucible is subjected to a first temperature increase ranging from −20° C. to 10° C., at a heating rate of 10° C./minute, and is then cooled from 100° C. to −20° C. at a cooling rate of 1° C./minute and is finally subjected to a second temperature increase ranging from −20° C. to 100° C. at a heating rate of 5° C./minute. During the second temperature increase, the variation of the difference in power absorbed by the empty crucible and by the crucible containing the sample of wax is measured as a function of the temperature. The melting point of the compound is the value of the temperature corresponding to the top of the peak of the curve representing the variation of the difference in power absorbed as a function of the temperature.

As used herein, the term “apolar wax” means a hydrocarbon-based or silicone apolar wax.

The waxes may be hydrocarbon-based waxes, fluoro waxes and/or silicone waxes and may be of plant, mineral, animal and/or synthetic origin. For example, the waxes have a melting point of greater than 45° C.

As wax that may be used in the composition of the present disclosure, non-limiting mention may be made of beeswax, carnauba wax or candelilla wax, paraffin, microcrystalline waxes, ceresin or ozokerite; synthetic waxes, for instance polyethylene waxes or Fischer-Tropsch waxes, and silicone waxes, for instance alkyl or alkoxy dimethicones comprising from 16 to 45 carbon atoms.

The composition according to the present disclosure may comprise an apolar wax such as a hydrocarbon-based or silicone apolar wax.

The composition may, for example, comprise an apolar hydrocarbon-based wax. As used herein, the term “apolar hydrocarbon-based wax” means a wax comprising at least 95% by weight of chemical compounds free of polar groups. The “polar groups” are well known to those skilled in the art; they may be, for example, ionic or nonionic polar groups chosen from —COOH; —OH; ethylene oxide; propylene oxide; —PO₄; —NHR; —NR₁R₂ with R₁ and R₂ optionally forming a ring and chosen from linear and branched C₁ to C₂₀ alkyl or alkoxy radicals.

According to one embodiment, the hydrocarbon-based wax comprises at least 95% by weight of compounds free of hetero atoms. As used herein, the term “hetero atom” means an atom other than carbon or hydrogen.

According to one embodiment, the apolar hydrocarbon-based wax comprises at least 95% by weight of chemical compounds comprising carbon and hydrogen. These chemical compounds may be, for example, chosen from linear or branched saturated alkanes.

According to one embodiment, the apolar wax is chosen from linear hydrocarbon-based waxes.

The linear hydrocarbon-based waxes may be chosen from ethylene polymers and copolymers, linear paraffin waxes and Fischer-Tropsch waxes.

Non-limiting illustrations of hydrocarbon-based waxes that may be mentioned may be chosen from Fischer-Tropsch waxes, which are also known as polymethylene waxes. They correspond to the formula CₙH₂ₙ₊₂.

According to one embodiment of the present disclosure, the at least one wax according to the present disclosure is a polymethylene wax, for example the wax Cirebelle 505® manufactured by the company Sasol, with a melting point equal to 40° C.

The apolar wax may be a wax of polyoxyalkylenated silicone type, i.e. a silicone comprising at least one oxyalkylene group of the type (—CₓH₂ₓO)ₐ wherein x may range from 2 to 6 and a is greater than or equal to 2.

The oxyalkylenated silicones that may be suitable for the present disclosure may be chosen from the compounds of general formulae (I), (II), (III), and (IV): 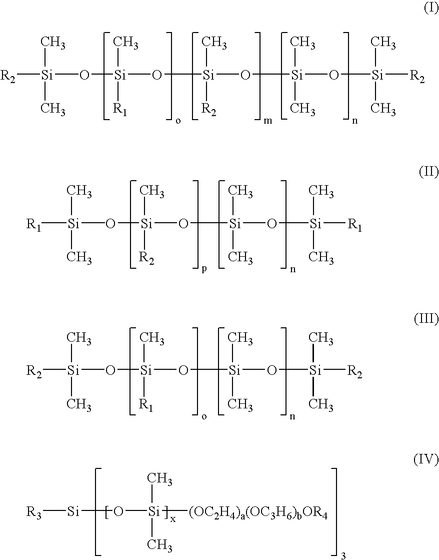
wherein:
R₁, which may be identical or different, is chosen from linear and branched C₁-C₃₀ alkyl and phenyl radicals,
R₂, which may be identical or different, is chosen from C_{c}H_{2c}—O—(C₂H₄O)ₐ(C₃H₆O)_{b}—R₅ and —C_{c}H_{2c}—O—(C₄H₈O)ₐ—R₅,
R₃ and R₄, which may be identical or different, are linear and branched C₁ to C₁₂ alkyl radicals, for example, a methyl radical,
R₅, which may be identical or different, is chosen from hydrogen atoms, linear and branched alkyl radicals of 1 to 12 carbon atoms, linear and branched alkoxy radicals of 1 to 6 carbon atoms, a linear and branched acyl radicals of 2 to 30 carbon atoms, a hydroxyl radicals, C₁-C₆ aminoalkoxy radicals optionally substituted on the amine, C₂-C₆ aminoacyl radicals optionally substituted on the amine, aminoalkyl radicals optionally substituted on the amine and on the alkyl chain, C₂-C₃₀ carboxyacyl radicals, groups optionally substituted with one or two substituted aminoalkyl radicals, —NHCO(CH₂)_{d}OH, a phosphate group,
R₇ is chosen from a hydrogen atom,
d ranges from 1 to 10,
m ranges from 0 to 20,
n ranges from 0 to 500,
o ranges from 0 to 20,
p ranges from 1 to 50,
a ranges from 0 to 50,
b ranges from 0 to 50,
a+b is greater than or equal to 2,
c ranges from 0 to 4,
x ranges from 1 to 100.

Such silicones are described, for example, in U.S. Pat. No. 5,070,171, U.S. Pat. No. 5,149,765, U.S. Pat. No. 5,093,452 and U.S. Pat. No. 5,091,493.

The silicones may be, for example, those of formula (III) wherein R₂, which may be identical or different, is chosen from C_{c}H_{2c}—O—(C₂H₄O)ₐ(C₃H₆O)_{b}—R₅, wherein R₅, a, b and c are defined as above. In this embodiment, b and c may be, for example, equal to 0 and a ranges from 1 to 50, for example, from 5 to 30 and further, by example, from 10 to 20.

The low-melting apolar wax may be, for example, present in an amount ranging from 1% to 30%, for example, from 3% to 20% by weight, relative to the total weight of the composition.

The mass ratio between the polymer particles and the low-melting apolar wax may, for example, range from 0.5 to 100, for example, from 1 to 50, and further, for example, from 1 to 20 and even further, for example, from 3 to 15.

The composition according to the present disclosure may also comprise at least one additional wax other than the low-melting apolar wax described above.

The at least one additional wax may have a melting point of greater than or equal to 65° C. It may be chosen from beeswax, carnauba wax, candelilla wax, paraffin, microcrystalline waxes, ceresin and ozokerite; polyethylene waxes, Fischer-Tropsch waxes; and mixtures thereof.

According to one embodiment, the apolar wax with a melting point of less than 65° C. (a) and the wax whose melting point is greater than or equal to 65° C. (b) are in a mass proportion (a)/(b) ranging from 30/70 to 55/45, for example, ranging from 40/60 to 45/55.

The amount of all the wax(es) comprised in the composition may, for example, range from 15% to 35%, for example, from 20% to 30% by weight, relative to the total weight of the composition.

The at least one additional wax may be, for example, an apolar wax with a melting point of greater than 65° C., for instance a microcrystalline wax, a polyethylene wax or a paraffin wax, or a mixture thereof.

The composition according to the present disclosure may also comprise a pasty compound.

Synthesis Medium for the Polymer Particles

According to one embodiment, the fatty phase of the composition comprises at least one oil, which is the organic solvent(s) serving as the polymerization medium for the polymer particles as described above.

The polymer dispersion may be manufactured as described in document EP-A-749 747.

A mixture comprising the initial monomers and also a free-radical initiator is prepared. This mixture is dissolved in a solvent, which is referred to in the description hereinbelow as the “synthesis solvent”.

A synthesis solvent is chosen wherein the initial monomers and the free-radical initiator are soluble therein, and the polymer particles obtained are insoluble therein, wherein they precipitate therefrom during their formation. For example, at least one synthesis solvent may be used and it may be chosen from alkanes such as heptane, isododecane or cyclohexane. The polymerization of the polymer particles may be performed in a synthesis solvent as described above, and the non-volatile hydrocarbon-based oil described above may then be added and the synthesis solvent may be selectively distilled off, provided that the non-volatile hydrocarbon-based oil is miscible with the synthesis solvent.

The monomers may, for example, be present in the synthesis solvent, before polymerization, in a proportion of 5-20% by weight of the reaction mixture. The total amount of the monomers may be present in the solvent before the start of the reaction, or some of the monomers may be added gradually as the polymerization reaction proceeds.

The free-radical initiator may be, for example, azobisisobutyronitrile or tert-butylperoxy-2-ethyl hexanoate.

Hydrophilic Medium

The composition according to the present disclosure may comprise a hydrophilic medium comprising water or a mixture of water and of one or more hydrophilic organic solvent(s), for instance alcohols, for example, linear and branched lower monoalcohols comprising from 2 to 5 carbon atoms, for instance ethanol, isopropanol or n-propanol, and polyols, for instance glycerol, diglycerol, propylene glycol, sorbitol, pentylene glycol and polyethylene glycols, or hydrophilic C₂ ethers and C₂-C₄ aldehydes.

The water or the mixture of water and of hydrophilic organic solvents may be present in the composition according to the present disclosure in an amount ranging from 0.1% to 99% by weight, for example, from 10% to 80% by weight, relative to the total weight of the composition.

At Least One Semi-Crystalline Polymer

In one embodiment, the composition of the present disclosure comprises at least one semi-crystalline polymer.

As used herein, the term “polymer” means compounds comprising at least two repeating units, for example, at least three repeating units, further, for example at least ten or more repeating units.

As used herein, the term “semi-crystalline polymer” means polymers comprising a crystallizable portion and an amorphous portion in the skeleton and a first-order reversible change of phase temperature, for example, of melting (solid-liquid transition). The crystallizable portion may be either a side chain (or pendent chain) or a block in the skeleton.

When the crystallizable portion of the semi-crystalline polymer is a block of the polymer skeleton, this crystallizable block may have a different chemical nature from that of the amorphous blocks; in this case, the semi-crystalline polymer is a block copolymer, for example of the diblock, triblock or multiblock type. When the crystallizable portion is a chain that is pendent on the skeleton, the semi-crystalline polymer may be a homopolymer or a copolymer.

As used herein, the terms “organic compound” and “an organic structure” mean compounds comprising carbon atoms and hydrogen atoms and optionally hetero atoms chosen from, for example, S, O, N and P, alone or in combination.

The melting point of the at least one semi-crystalline polymer may be, for example, less than 150° C.

The melting point of the at least one semi-crystalline polymer may be, for example, greater than or equal to 30° C. and less than 100° C. For example, the melting point of the at least one semi-crystalline polymer may be greater than or equal to 30° C. and less than 60° C.

The at least one semi-crystalline polymer may be solid at room temperature (25° C.) and atmospheric pressure (760 mmHg), with a melting point of greater than or equal to 30° C. The melting point values correspond to the melting point measured using a differential scanning calorimeter (DSC), such as the calorimeter sold under the name DSC 30 by the company Mettler, with a temperature rise of 5 or 10° C. per minute (the melting point under consideration is the point corresponding to the temperature of the most endothermic peak of the thermogram).

The at least one semi-crystalline polymer according to the present disclosure may have a melting point that is higher than the temperature of the keratinous support intended to receive the composition, for example, the skin or the lips.

The at least one semi-crystalline polymer according to the present disclosure is capable, alone or as a mixture, of structuring the composition without the addition of a surfactant and/or a filler and/or a wax.

According to the present disclosure, the at least one semi-crystalline polymer may be, for example, soluble in the at least one fatty phase, for example, to at least 1% by weight, at a temperature that is higher than their melting point. Besides the crystallizable chains or blocks, the blocks of the polymers may be amorphous.

For the purposes of the present disclosure, the expression “crystallizable chain or block” means a chain or block which, if it were obtained alone, would change from the amorphous state to the crystalline state reversibly, depending on whether one is above or below the melting point. For the purposes of the present disclosure, a “chain” is a group of atoms, which are pendent or lateral relative to the polymer skeleton. As used herein, the term “block” is a group of atoms belonging to the skeleton, this group constituting one of the repeating units of the polymer.

For example, the polymer skeleton of the at least one semi-crystalline polymer is soluble in the at least one fatty phase.

For example, the crystallizable blocks or chains of the at least one semi-crystalline polymer represent at least 30% of the total weight of each polymer, for example, at least 40%. The at least one semi-crystalline polymer comprising crystallizable side chains are homopolymers or copolymers. The at least one semi-crystalline polymer of the present disclosure comprising crystallizable blocks are block or multiblock copolymers. They may be obtained by polymerizing a monomer comprising reactive (or ethylenic) double bonds or by polycondensation. When the polymers of the present disclosure are polymers comprising crystallizable side chains, these side chains may be, for example, in random or statistical form.

For example, the at least one semi-crystalline polymer of the present disclosure is of synthetic origin. According to one embodiment of the present disclosure, the at least one semi-crystalline polymer of the present disclosure do not comprise a polysaccharide skeleton.

The at least one semi-crystalline polymer that may be used in the present disclosure may, for example, be chosen from:
block copolymers of polyolefins of controlled crystallization, whose monomers are described in EP-A-0 951 897,
polycondensates, for example, of aliphatic or aromatic polyester type or of aliphatic/aromatic polyester type,
homopolymers or copolymers bearing at least one crystallizable side chain and homopolymers or copolymers bearing at least one crystallizable block in the skeleton, for instance those described in document U.S. Pat. No. 5,156,911,
homopolymers or copolymers bearing at least one crystallizable side chain, for example, bearing fluoro group(s), such as those described in document WO-A-01/19333,
and mixtures thereof.

In the last two cases, the crystallizable side chain(s) or block(s) is (are) hydrophobic.

A) At Least One Semi-Crystalline Polymers Comprising Crystallizable Side Chains

Non-limiting mention may be made, for example, of those defined in documents U.S. Pat. No. 5,156,911 and WO-A-01/19333. They are homopolymers or copolymers comprising from 50% to 100% by weight of units resulting from the polymerization of at least one monomers bearing a crystallizable hydrophobic side chain.
These homopolymers or copolymers are of any nature, provided that they meet the conditions mentioned hereinbelow, for example, may be soluble or dispersible in the fatty phase, by heating above their melting point (mp). They can result:
from the polymerization, for example, the free-radical polymerization, of at least one monomers comprising reactive or ethylenic double bond(s) with respect to a polymerization, namely a vinyl, (meth)acrylic or allylic group,
from the polycondensation of at least one monomers bearing co-reactive groups (carboxylic acid, sulfonic acid, alcohol, amine or isocyanate), such as, for example, polyesters, polyurethanes, polyethers, polyureas or polyamides.
a) In general, the crystallizable units (chains or blocks) of the at least one semi-crystalline polymer according to the present disclosure are derived from monomers comprising crystallizable blocks or chains, used for manufacturing semi-crystalline polymers. These polymers may be chosen, for example, from homopolymers and copolymers resulting from the polymerization of at least one monomer comprising crystallizable chain(s) that may be represented by formula (X): 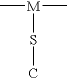
   wherein M is an atom of the polymer skeleton,
   S is a spacer and
   C is a crystallizable group

The crystallizable chains “—S—C” may be aliphatic or aromatic, and optionally fluorinated or perfluorinated. “S”, for example, may be a group chosen from (CH₂)ₙ (CH₂CH₂O)ₙ and (CH₂O), which may be chosen from linear, branched and cyclic, wherein n is an integer ranging from 0 to 22. For example, “S” is a linear group. For example, “S” and “C” are different.

When the crystallizable chains are hydrocarbon-based aliphatic chains, they comprise hydrocarbon-based alkyl chains comprising at least 11 carbon atoms and not more than 40 carbon atoms, for example, not more than 24 carbon atoms. They are, for example, aliphatic chains or alkyl chains comprising at least 12 carbon atoms, and they may be C₁₄-C₂₄, for example, C₁₆-C₂₂, alkyl chains. When they are fluoroalkyl or perfluoroalkyl chains, they comprise at least 11 carbon atoms, at least 6 of which carbon atoms are fluorinated.

As examples of semi-crystalline homopolymers or copolymers comprising crystallizable chain(s), non-limiting mention may be made of those resulting from the polymerization of at least one of the following monomers: (meth)acrylates of saturated alkyls with the alkyl group chosen from C₁₄-C₂₄, perfluoroalkyl (meth)acrylates with a C₁₁-C₁₅ perfluoroalkyl group, N-alkyl(meth)acrylamides with the alkyl group chosen from C₁₄ to C₂₄ with or without a fluorine atom, vinyl esters comprising alkyl or perfluoroalkyl chains with the alkyl group chosen from C₁₄ to C₂₄ (with at least 6 fluorine atoms per perfluoroalkyl chain), vinyl ethers comprising alkyl or perfluoroalkyl chains with the alkyl group chosen from C₁₄ to C₂₄ and at least 6 fluorine atoms per perfluoroalkyl chain, C₁₄ to C₂₄ α-olefins such as, for example, octadecene, para-alkylstyrenes with an alkyl group comprising from 12 to 24 carbon atoms, and mixtures thereof.

When the polymers result from a polycondensation, the hydrocarbon-based and/or fluorinated crystallizable chains as defined above are borne by a monomer that may be a diacid, a diol, a diamine or a diisocyanate.

In another embodiment of the invention, when the polymers are copolymers, they may, for example, range from 0 to 50% of groups Y or Z resulting from the copolymerization:
1) of Y which is a polar or non-polar monomer or a mixture of the two:
   Wherein Y is a polar monomer, it is either a monomer bearing polyoxyalkylenated groups (especially oxyethylenated and/or oxypropylenated groups), a hydroxyalkyl (meth)acrylate, for instance hydroxyethyl acrylate, (meth)acrylamide, an N-alkyl(meth)acrylamide, an N,N-dialkyl(meth)acrylamide such as, for example, N,N-diisopropylacrylamide or N-vinylpyrrolidone (NVP), N-vinylcaprolactam, a monomer bearing at least one carboxylic acid group, for instance (meth)acrylic acid, crotonic acid, itaconic acid, maleic acid or fumaric acid, or bearing a carboxylic acid anhydride group, for instance maleic anhydride, and mixtures thereof.
   Wherein Y is a non-polar monomer, it may be an ester of the linear, branched or cyclic alkyl (meth)acrylate type, a vinyl ester, an alkyl vinyl ether, an α-olefin, styrene or styrene substituted with a C₁ to C₁₀ alkyl group, for instance α-methylstyrene, or a macromonomer of the polyorganosiloxane type comprising vinyl unsaturation.

As used herein, the term “alkyl” means a saturated group, for example, of C₈ to C₂₄, except where otherwise mentioned.
2) of Z which is a polar monomer or a mixture of polar monomers. In this case, Z is either a monomer bearing polyoxyalkylenated groups (especially oxyethylenated and/or oxypropylenated groups), a hydroxyalkyl (meth)acrylate, for instance hydroxyethyl acrylate, (meth)acrylamide, an N-alkyl(meth)acrylamide, an N,N-dialkyl(meth)acrylamide such as, for example, N,N-diisopropylacrylamide or N-vinylpyrrolidone (NVP), N-vinylcaprolactam, a monomer bearing at least one carboxylic acid group, for instance (meth)acrylic acid, crotonic acid, itaconic acid, maleic acid or fumaric acid, or bearing a carboxylic acid anhydride group, for instance maleic anhydride, and mixtures thereof.

For example, the at least one semi-crystalline polymer comprising a crystallizable side chain may be chosen from alkyl (meth)acrylate or alkyl(meth)acrylamide homopolymers with an alkyl group as defined above, and, for example, of C₁₄-C₂₄, copolymers of these monomers with a hydrophilic monomer, for example, of a different nature from (meth)acrylic acid, for instance N-vinylpyrrolidone or hydroxyethyl (meth)acrylate, and mixtures thereof.

In an embodiment, the at least one semi-crystalline polymer comprising a crystallizable side chain has a weight-average molar mass (Mp) ranging from 5000 to 1,000,000, for example, from 10,000 to 800,000, further, for example, from 15,000 to 500,000 and even further, for example, from 0.100,000 to 200,000.

An example of the at least one semi-crystalline polymer that may be used in the composition according to the present disclosure, non-limiting mention may be made of the Intelimer® products from the company Landec described in the brochure “Intelimer® polymers”, Landec IP22 (Rev. 4-97). These polymers are in solid form at room temperature (25° C.). They bear crystallizable side chains and have the above formula X.

For example, the Intelimer® product IPA 13-1 from the company Landec may be used, which is a polystearyl acrylate with a molecular weight of 145,000 and a melting point of 49° C.

The at least one semi-crystalline polymer may, for example, be those described in Examples 3, 4, 5, 7 and 9 of U.S. Pat. No. 5,156,911, comprising a —COOH group, resulting from the copolymerization of acrylic acid and of a C₅ to C₁₆ alkyl (meth)acrylate with a melting point ranging from 20° C. to 35° C., and, for example, from the copolymerization:
of acrylic acid, of hexadecyl acrylate and of isodecyl acrylate in a 1/16/3 ratio,
of acrylic acid and of pentadecyl acrylate in a 1/19 ratio,
of acrylic acid, of hexadecyl acrylate and of ethyl acrylate in a 2.5/76.5/20 ratio,
of acrylic acid, of hexadecyl acrylate and of methyl acrylate in a 5/85/10 ratio,
of acrylic acid and of polyoctadecyl (meth)acrylate in a 2.5/97.5 ratio.

It is also possible to use the polymer Structure “O” from National Starch, such as the product described in document U.S. Pat. No. 5,736,125 with a melting point of 44° C.

The at least one semi-crystalline polymer may, for example, be a semi-crystalline polymer with crystallizable pendent chains comprising fluoro groups, as described in Examples 1, 4, 6, 7 and 8 of document WO-A-01/19333.

It is also possible to use semi-crystalline polymers obtained by copolymerization of stearyl acrylate and of acrylic acid or of NVP, as described in document U.S. Pat. No. 5,519,063 or EP-A-550 745.

It is also possible to use semi-crystalline polymers obtained by copolymerization of behenyl acrylate and of acrylic acid or of NVP, as described in documents U.S. Pat. No. 5,519,063 and EP-A-550 745 and, for example, those described in Examples 3 and 4 below, of polymer preparation.

B) At Least One Polymer Bearing at Least One Crystallizable Block in the Skeleton

In one embodiment, the composition disclosed herein comprises at least one polymer that is soluble or dispersible in the at least one fatty phase by heating above its melting point (mp). This polymer may be chosen from, for example, block copolymers comprising at least two blocks of different chemical nature, one of which is crystallizable.

The at least one polymer bearing at least one crystallizable block in the skeleton may be chosen from block copolymers of olefin or of cycloolefin comprising a crystallizable chain, for instance those derived from the block polymerization of:
cyclobutene, cyclohexene, cyclooctene, norbornene (i.e. bicyclo(2,2,1)-2-heptene), 5-methylnorbornene, 5-ethylnorbornene, 5,6-dimethylnorbornene, 5,5,6-trimethylnorbornene, 5-ethylidenenorbornene, 5-phenylnorbornene, 5-benzylnorbornene, 5-vinylnorbornene, 1,4,5,8-dimethano-1,2,3,4,4a,5,8a-octahydronaphthalene, dicyclopentadiene, or mixtures thereof,
with ethylene, propylene, 1-butene, 3-methyl-1-butene, 1-hexene, 4-methyl-1-pentene, 1-octene, 1-decene or 1-eicosene, or mixtures thereof,
and, for example, copoly(ethylene/norbornene) blocks and (ethylene/propylene/ethylidene-norbornene) block terpolymers. Those resulting from the block copolymerization of at least 2 C₂-C₁₆, for example, C₂-C₁₂ α-olefins such as those mentioned above and block bipolymers of ethylene and of 1-octene may also be used.

The at least one polymer bearing at least one crystallizable block in the skeleton may be chosen from copolymers comprising at least one crystallizable block, the rest of the copolymer is amorphous (at room temperature). These copolymers may also comprise two crystallizable blocks of different chemical nature.

Other copolymers which may be used, for example, according to the disclosure are those that simultaneously comprise at room temperature a crystallizable block and an amorphous block that are both hydrophobic and lipophilic, sequentially distributed; non-limiting mention may be made, for example, of polymers comprising one of the crystallizable blocks and one of the amorphous blocks below:
Blocks that is crystallizable by nature, of polyester type, for instance poly(alkylene terephthalate), or of polyolefin type, for instance polyethylenes or polypropylenes.
Amorphous and lipophilic blocks, for instance: amorphous polyolefins or copoly(olefin)s such as poly(isobutylene), hydrogenated polybutadiene or hydrogenated poly(isoprene).

As examples of such copolymers comprising a crystallizable block and an amorphous block, non-limiting mention may be made of:
1) poly(ε-caprolactone)-b-poly(butadiene) block copolymers, for example, used hydrogenated, such as those described in the article “Melting behavior of poly(-caprolactone)-block-polybutadiene copolymers” from S. Nojima, Macromolecules, 32, 3727-3734 (1999),
2) the hydrogenated block or multiblock poly(butylene terephthalate)-b-poly(isoprene) block copolymers cited in the article “Study of morphological and mechanical properties of PP/PBT” by B. Boutevin et al., Polymer Bulletin, 34, 117-123 (1995),
3) the poly(ethylene)-b-copoly(ethylene/propylene) block copolymers cited in the articles “Morphology of semi-crystalline block copolymers of ethylene-(ethylene-alt-propylene)” by P. Rangarajan et al., Macromolecules, 26, 4640-4645 (1993) and “Polymer aggregates with crystalline cores: the system poly(ethylene)-poly(ethylene-propylene)” by P. Richter et al., Macromolecules, 30, 1053-1068 (1997),
4) the poly(ethylene)-b-poly(ethylethylene) block copolymers cited in the general article “Crystallization in block copolymers” by I. W. Hamley, Advances in Polymer Science, Vol. 148, 113-137 (1999).
   C) Polycondensates of Aliphatic or Aromatic or Aliphatic/Aromatic Polyester Type

In one embodiment, the composition according to the disclosure may comprise at least one polyester polycondenstate. The polyester polycondensates may be chosen from aliphatic polyesters. Their molar mass may be, for example, greater than or equal to 200 and less than or equal to 10,000, and further, for example, greater than or equal to 300 and less than or equal to 5000, even further, for example, greater than or equal to 500 and less than or equal to 2000 g/mol.

The polyester polycondensates are, for example, chosen from polycaprolactone. For example, the polycaprolactones may be chosen from ε-caprolactone homopolymers. The homopolymerization may be initiated with, for example, a diol, such as a diol comprising from 2 to 10 atoms, such as diethylene glycol, 1,4-butanediol or neopentyl glycol.

Polycaprolactones that may be used, for example, are those sold under the name CAPA® 240 (melting point of 68° C. and molecular weight of 4000), CAPA®223 (melting point of 48° C. and molecular weight of 2000), CAPA®222 (melting point of 48° C. and molecular weight of 2000), CAPA®217 (melting point of 44° C. and molecular weight of 1250), CAPA®2125 (melting point of 45° C. and molecular weight of 1250), CAPA®212 (melting point of 45° C. and molecular weight of 1000), CAPA®210 (melting point of 38° C. and molecular weight of 1000), CAPA®205 (melting point of 39° C. and molecular weight of 830) by the company Solvay, or PCL-300 and PCL-700 by the company Union Carbide.

The CAPA® 2125 whose melting point ranges from 35 to 45° C. and whose molecular weight is equal to 1250 may be used.

The at least one semi-crystalline polymer in the composition of the present disclosure may or may not be partially crosslinked, provided that the degree of crosslinking does not interfere with their dissolution or dispersion in the at least one fatty phase by heating above their melting point. It may then be a chemical crosslinking, by reaction with a multifunctional monomer during the polymerization. It may also be a physical crosslinking which may, in this case, be due either to the establishment of bonds of hydrogen or dipolar type between groups borne by the polymer, such as, for example, the dipolar interactions between carboxylate ionomers, these interactions are of small amount and borne by the polymer skeleton; or to a phase separation between the crystallizable blocks and the amorphous blocks borne by the polymer.

For example, the at least one semi-crystalline polymer in the composition according to the present disclosure is non-crosslinked.

In practice, the total amount of semi-crystalline polymer(s) is present in an amount ranging from 0.1% to 80% of the total weight of the composition, from 0.5% to 40% and from 3% to 30% of the total weight of the composition. It may, for example, be present in an amount ranging from 5% to 25% by weight of the composition.

Particulate Phase

In one embodiment, the composition according to the present disclosure may comprise at least one pigments and/or at least one fillers.

The pigments may be white or colored, mineral and/or organic, and coated or uncoated. Among the mineral pigments which may be mentioned are titanium dioxide, optionally surface-treated, zirconium oxide or cerium oxide, as well as zinc oxide, iron oxide (black, yellow or red), chromium oxide, manganese violet, ultramarine blue, chromium hydrate and ferric blue. Among the organic pigments that may be mentioned are carbon black, pigments of the type such as organic lakes of barium, strontium, calcium or aluminium, for example, those submitted for certification by the Food and Drug Administration (FDA) (e.g. D&C or FD&C) and those exempt from FDA certification, for instance lakes based on cochineal carmine. The pigments may, for example, be present in an amount ranging from 0.1% to 50%, for example, from 0.5% to 35% and further, for example, ranging from 2% to 25% of active material, relative to the total weight of the composition.

The nacreous pigments may be chosen from white nacreous pigments such as mica coated with titanium or with bismuth oxychloride, colored nacreous pigments such as titanium mica with iron oxides, titanium mica with, for example, ferric blue or chromium oxide, titanium mica with an organic pigment of the type mentioned above, as well as nacreous pigments based on bismuth oxychloride. They may optionally represent from 0 to 25% (of active material), for example, from 0.1% to 15% of the total weight of the composition (if present). Pigments with goniochromatic properties and/or pigments with a metallic effect as described in the French Patent Application No. FR 2 842 417, the content of which is incorporated into the present patent application by reference, may thus be used.

The fillers may be mineral or organic, and lamellar or spherical. Non-limiting mention may be made of talc, mica, silica, kaolin, Nylon® powder (Orgasol® from Atochem), poly-β-alanine powder and polyethylene powder, powders of tetrafluoroethylene polymers (Teflon®)), lauroyllysine, starch, boron nitride, hollow microspheres such as Expancel®) (Nobel Industrie), Polytrap® (Dow Corning) and silicone resin microbeads (for example Tospearls® from Toshiba), precipitated calcium carbonate, magnesium carbonate, magnesium hydrocarbonate, hydroxyapatite, hollow silica microspheres (Silica Beads® from Maprecos), glass or ceramic microcapsules, metal soaps derived from carboxylic organic acids comprising from 8 to 22 carbon atoms, for example, from 12 to 18 carbon atoms, for example zinc stearate, magnesium stearate or lithium stearate, zinc laurate or magnesium myristate.

The composition according to the present disclosure, for example, comprises particles that are solid at room temperature, dispersed in the physiologically acceptable medium, introduced into the composition in the form of a colloidal dispersion, as described in PCT Patent Application No. WO 02/39961, the content of which is herein incorporated by reference.

The composition may comprise at least one dispersant. The dispersant may serve to protect the dispersed filler or pigment particles against their agglomeration or flocculation. The concentration of dispersant used, for example, to stabilize a colloidal dispersion ranges from 0.3 to 5 mg/m², for example, from 0.5 to 4 mg/m² of surface area of pigment and/or filler particles. This at least one dispersant may be a surfactant, an oligomer, a polymer or a mixture of several thereof, comprising at least one functionalities with strong affinity for the surface of the particles to be dispersed. For example, they may optionally physically or chemically attach to the surface of the pigments. These dispersants also have at least one functional group that is compatible with or soluble in the continuous medium. For example, esters of 12-hydroxystearic acid, for example, and of a C₈ to C₂₀ fatty acid and of a polyol, for instance glycerol or diglycerol are used, such as poly(12-hydroxystearic acid) stearate with a molecular weight of 750 g/mol, such as the product sold under the name Solsperse 21,000 by the company Avecia, polyglyceryl-2 dipolyhydroxystearate (CTFA name) sold under the reference Dehymyls PGPH by the company Henkel, or polyhydroxystearic acid such as the product sold under the reference Arlacel P100 by the company Uniqema, and mixtures thereof.

As other dispersants that may be used in the composition of the present disclosure, non-limiting mention may be made of quaternary ammonium derivatives of polycondensed fatty acids, for instance Solsperse 17,000 sold by the company Avecia, and mixtures of polydimethylsiloxane/oxypropylene such as those sold by the company Dow Corning under the references DC2-5185 and DC2-5225 C.

The polydihydroxystearic acid and the 12-hydroxystearic acid esters are, for example, intended for a hydrocarbon-based or fluorinated medium, whereas the mixtures of oxyethylene/oxypropylene dimethylsiloxane are, for example, intended for a silicone medium.

The colloidal dispersion is a suspension of particles, for example, of micron size (<10 μm) in a continuous medium. The volume fraction of particles in a concentrated dispersion ranges from 20% to 40%, for example, greater than 30%, which corresponds to a weight content that may be up to 70% according to the density of the particles.

Additives and Galenical Forms

In one embodiment, the composition of the present disclosure may also comprise at least one cosmetic or dermatological active agents such as those conventionally used.

As cosmetic, dermatological, hygiene or pharmaceutical active agents that may be used in the composition of the present disclosure, non-limiting mention may be made of moisturizers, vitamins, essential fatty acids, sphingolipids and sunscreens. These active agents are used in a usual amount for a person skilled in the art, for example, at concentrations of from 0 to 20% and further, for example, from 0.001% to 15% of the total weight of the composition.

The composition may also comprise any other additive usually used in such compositions, such as water, gelling agents, water-soluble dyes, antioxidants, fragrances, preserving agents and essential oils.

Needless to say, a person skilled in the art will take care to select this or these optional additional compound(s), and/or the amount thereof, so that the advantageous properties of the composition according to the present disclosure are not, or are not substantially, adversely affected by the envisaged addition.

In one embodiment of the present disclosure, the compositions according to the present disclosure may be prepared in the usual manner by a person skilled in the art. They may be in the form of a cast product, for example in the form of a stick or tube, or in the form of a dish that may be used by direct contact or with a sponge. For example, they find an application as a cast foundation, a cast makeup rouge, a cast eyeshadow, a lipstick, base or balm to care for the lips, or a concealer product. They may also be in the form of a soft paste or a more or less fluid gel or cream, or a liquid, packaged in a tube. They may then constitute foundations, lipsticks, antisun products or skin-coloring products.

The composition of the present disclosure may be anhydrous, for example, comprise less than 5% of water relative to the total weight of the composition.

These compositions for topical application may constitute, for example, a cosmetic, dermatological, hygiene or pharmaceutical composition for protecting, treating or caring for the face, the neck, the hands or the body (for example care cream, antisun oil or body gel), a makeup composition (for example a makeup gel, cream or stick) or a composition for artificially tanning or for protecting the skin.

The composition according to the present disclosure may be in the form of a dermatological or care composition for the skin and/or the integuments or in the form of an antisun composition or a body hygiene composition, for example, in deodorant form. It may be, for example, in uncolored form. It may then be used as a care base for the skin, the integuments or the lips (lip balms, for protecting the lips against the cold and/or sunlight and/or the wind, or a care cream for the skin, the nails or the hair).

The compositions of the present disclosure may be obtained by heating the various constituents to the melting point of the highest-melting waxes, followed by casting of the molten mixture in a mold (dish or finger stall). They may also be obtained by extrusion, as described in European Patent Application No. EP-A-0 667 146.

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the embodiments disclosed herein. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosed embodiments are approximations, unless otherwise indicated the numerical values set forth in the specific examples are reported as precisely as possible. Any numerical value, however, inherently contain certain errors necessarily resulting from the standard deviation found in their respective testing measurements.

The embodiments disclosed herein are illustrated in greater detail by the examples described below.

### EXAMPLE 1

### Lipstick

| | Mass |
|---|---|
| Chemical name | percentage |
| Dispersion of acrylate polymer in | 30 |
| hydrogenated polyisobutene, surface- | |
| stabilized with Kraton G1701 | |
| 2-Decyltetradecanoic acid triglyceride | 2.02 |
| Dilinoleyl diol dimer/dilinoleic dimer | 10 |
| copolymer (Lusplan DD-DA5 ®) | |
| Octyldodecanol | 9 |
| Preserving agent | 0.47 |
| Polycaprolactone of MW 1250 g/mol | 9 |
| Vinylpyrrolidone/eicosene copolymer | 6 |
| Microcrystalline wax | 10 |
| Polyethylene wax (MW 500) | 2 |
| Polymethylene wax of m.p. 40° C. | 10 |
| Stearyl alcohol | / |
| Pigments | 6.03 |
| Dimethicone-coated silica | 5 |
| Fragrance | 0.48 |
| TOTAL | 100 |

Synthesis of the Dispersion of Polymer Particles:

A dispersion of non-crosslinked copolymer of methyl acrylate and of acrylic acid in an 85/15 ratio, in heptane, was prepared according to the method of Example 1 of document EP-A-749 746. When the polymerization was complete, hydrogenated polyisobutene was added and the heptane was distilled off under vacuum.

A dispersion of poly(methyl acrylate/acrylic acid) particles stabilized in the hydrogenated polyisobutene with a polystyrene/copoly(ethylene-propylene) diblock copolymer sold under the name Kraton G 1701, with a solids content of 21% by weight and a mean particle size equal to 150 nm, was obtained.

Procedure for Preparing the Lipstick

All the starting materials were weighed out in an oil-circulated jacketed heating pan and were then heated with stirring (turbomixer).

After total melting of the materials and homogenization of the mixture, it was ground 5 times in succession on a three-roll mill. The paste obtained was left to stabilize for 24 hours at 20° C. and then packaged in heating bags.

What is claimed is:

1. A cosmetic composition comprising particles of at least one polymer dispersed in at least one fatty phase,
wherein the at least one fatty phase comprises no more than 50% by weight of volatile oil, relative to the weight of the at least one fatty phase, and
wherein the at least one fatty phase comprises at least 5% by weight of at least one sparingly polar or apolar oil, relative to the weight of the at least one fatty phase.

2. A composition according to claim 1, wherein the at least one fatty phase comprises at least 10% by weight of at least one apolar or sparingly polar oil.

3. A composition according to claim 1, wherein the at least one fatty phase comprises no more than 40% by weight of at least one volatile oil, relative to the total weight of the fatty phase.

4. A composition according to claim 3, wherein the at least one fatty phase comprises no more than 10% by weight of at least one volatile oil, relative to the total weight of the at least one fatty phase.

5. A composition according to claim 4, wherein the composition is free of volatile oil.

6. A composition according to claim 1, wherein the mean gloss at 20° of a deposit of the composition spread onto a support is no less than 30 out of 100.

7. A composition according to claim 6, wherein the mean gloss is no less than 35 out of 100.

8. A composition according to claim 7, wherein the mean gloss is no less than 60 out of 100.

9. A composition according to claim 1, wherein the mean gloss at 60° of a deposit of the composition spread onto a support is no less than 50 out of 100.

10. A composition according to claim 10, wherein the mean gloss at 60° of a deposit of the composition spread onto a support is no less than 90 out of 100.

11. A composition according to claim 1, wherein the mean gloss at 20° of a deposit of the composition spread onto a support is no less than 35 out of 100 and the mean gloss at 60° of a deposit of the composition spread onto a support is no less than 65 out of 100.

12. A composition according to claim 1, wherein the particles of at least one polymer are solid and insoluble in the at least one fatty phase at a temperature of 25° C.

13. A composition according to claim 1, wherein the at least one polymer is not a wax.

14. A composition according to claim 1, wherein the particles of at least one polymer have a mean size ranging from 5 to 800 nm.

15. A composition according to claim 1, wherein the at least one polymer is film-forming.

16. A composition according to claim 15, wherein the at least one polymer is a hydrocarbon-based polymer.

17. A composition according to claim 1, wherein the particles of at least one polymer are insoluble in water-soluble alcohols.

18. A composition according to claim 1, wherein the at least one polymer is chosen from polyurethanes, polyurethane-acrylics, polyureas, polyureas/polyurethanes, polyester-polyurethanes, polyether-polyurethanes, polyesters, polyester amides, fatty-chain polyesters, alkyds; acrylic and/or vinyl polymers or copolymers; acrylic-silicone copolymers; polyacrylamides; silicone polymers, and fluoro polymers.

19. A composition according to claim 1, wherein the at least one polymer is chosen from meth(acrylic)/methacrylate copolymers.

20. A composition according to claim 19, wherein the at least one polymer is chosen from acrylic/acrylate copolymers wherein the mass ratio of the acrylic units and of the acrylate units ranges from 0.1% to 40%.

21. A composition according to claim 20, wherein the at least one polymer is chosen from acrylic/acrylate copolymers wherein the mass ratio of the acrylic units and of the acrylate units ranges from 5% to 20%.

22. A composition according to claim 1, wherein the at least one polymer is present in an amount ranging from 5% to 40% of the total weight of the composition.

23. A composition according to claim 22, wherein the at least one polymer is present in an amount ranging from 8% to 30% of the total weight of the composition.

24. A composition according to claim 1, wherein the composition comprises at least one stabilizer chosen from block polymers, grafted polymers and random polymers.

25. A composition according to claim 24, wherein the at least one stabilizer is a grafted-block or block polymer comprising at least one block resulting from the polymerization of diene and at least one block of a vinyl polymer.

26. A composition according to claim 25, wherein the at least one stabilizer is a diblock polymer.

27. A composition according to claim 1, wherein the at least one apolar or sparingly polar oil is a hydrocarbon-based oil.

28. A composition according to claim 27, wherein the at least one apolar or sparingly polar oil is present in an amount ranging from 5% to 80% by weight, relative to the total weight of the composition.

29. A composition according to claim 28, wherein the at least one apolar or sparingly polar oil is present in an amount ranging from 15% to 30% by weight, relative to the total weight of the composition.

30. A composition according to claim 1, wherein the apolar or sparingly polar oil is a non-volatile hydrocarbon-based apolar oil.

31. A composition according to claim 30, wherein the non-volatile hydrocarbon-based apolar oil is chosen from oils with a molar mass ranging from 300 to 900 g/mol.

32. A composition according to claim 31, wherein the non-volatile hydrocarbon-based apolar oil is chosen from oils with a molar mass ranging from 350 to 800 g/mol.

33. A composition according to claim 31, wherein the non-volatile hydrocarbon-based apolar oil is chosen from linear or branched hydrocarbons chosen from liquid paraffin, liquid petroleum jelly and liquid naphthalene, hydrogenated polyisobutene, isoeicosane, squalane and decene/butene copolymers, and mixtures thereof.

34. A composition according to claim 1, wherein the at least one sparingly polar or apolar oil includes an oil of high molar mass in an amount ranging from 2% to 30%, relative to the total weight of the composition.

35. A composition according to claim 34, wherein the at least one sparingly polar or apolar oil includes an oil of high molar mass in an amount ranging from 5% to 15%, relative to the total weight of the composition.

36. A composition according to claim 1, wherein the at least one sparingly polar or apolar oil has a molar mass ranging from 650 to 10,000 g/mol.

37. A composition according to claim 36, wherein the at least one sparingly polar or apolar oil has a molar mass ranging from 750 to 7,500 g/mol.

38. A composition according to claim 1, comprising at least one pulverulent dyestuff chosen from pigments, nacres, and flakes.

39. A composition according to claim 38, comprising at least one dispersant chosen from poly(12-hydroxystearic acid stearate), poly(12-hydroxystearic acid) and diglyceryl 2-dipolyhydroxystearate.

40. A composition according to claim 1, wherein the composition is in anhydrous form.

41. A composition according to claim 1, wherein the composition is in a form of a product for caring for and/or making up the skin and/or the lips.

42. A composition according to claim 1, wherein the composition is in a form chosen from a foundation, a makeup rouge, an eyeshadow, a lipstick, a care base or care balm for the lips, a concealer product, an eyeliner or a mascara.

43. A composition according to claim 1, wherein the composition is in the form of a makeup which has at least one property chosen from glossy, non-migrating and good staying power.
